(19) United States
(12) Patent Application Publication
(10) Pub. No.: US 2024/0180228 A1
(43) Pub. Date: Jun. 6, 2024

(54) AEROSOLISABLE FORMULATION

(71) Applicant: RAI Strategic Holdings, Inc., Winston-Salem, NC (US)

(72) Inventors: John Will Caraway, JR., Clemmons, NC (US); Michael Foster Davis, Clemmons, NC (US); Jennifer Maria Rowe, Clemmons, NC (US); John Ulrich, Pittsboro, NC (US)

(21) Appl. No.: 18/043,869

(22) PCT Filed: Sep. 2, 2021
(86) PCT No.: PCT/IB2021/058006

§ 371 Date: Mar. 2, 2023

Related U.S. Application Data
(60) Provisional application No. 63/073,989, filed on Sep. 3, 2020.

(51) Int. Cl. A24B 15/167; A24B 15/38; A24F 40/10

(52) U.S. Cl. CPC A24B 15/167; A24B 15/385; A24F 40/10

(57) ABSTRACT

There is provided an aerosolisable formulation comprising (i) caffeine; (ii) water; and (iii) propylene glycol; wherein the weight ratio of water to propylene glycol is from 30:70 to 60:40.

### FIELD OF THE INVENTION

The present disclosure relates to an aerosolisable formulation, a method of forming the same, a container containing the same, a device containing the same and processes and uses of the same.

### BACKGROUND TO THE INVENTION

Aerosol delivery systems which generate an aerosol for inhalation by a user are known in the art. Such systems typically comprise an aerosol generator which is capable of converting an aerosolisable material into an aerosol. In some instances, the aerosol generated is a condensation aerosol whereby an aerosolisable material is heated to form a vapor which is then allowed to condense into an aerosol. In other instances, the aerosol generated is an aerosol which results from the atomization of the aerosolisable material. Such atomization may be brought about mechanically, e.g. by subjecting the aerosolisable material to vibrations so as to form small particles of material that are entrained in airflow. Alternatively, such atomization may be brought about electrostatically, or in other ways, such as by using pressure etc.

Depending on the constituents of the aerosolisable material that are to be provided to a user, it may be preferable to formulate the aerosolisable material in a certain way. For example, it may be preferable to formulate the aerosolisable material so as to produce an aerosol with a particular profile. It may also be preferable to formulate the aerosolisable material so as to ensure the aerosolisable material meets certain standards of quality, consistency and the like.

It would thus be desirable to provide an aerosolisable material that is formulated so as to be acceptable to a user

### SUMMARY OF THE INVENTION

In one aspect there is provided an aerosolisable formulation comprising (i) caffeine; (ii) water; and (iii) propylene glycol; wherein the weight ratio of water to propylene glycol is from 30:70 to 60:40.

In one aspect there is provided a process for forming an aerosol, the process comprising aerosolising an aerosolisable formulation comprising (i) caffeine; (ii) water; and (iii) propylene glycol; wherein the weight ratio of water to propylene glycol is from 30:70 to 60:40.

In one aspect there is provided a contained aerosolisable formulation comprising (a) a container; and (b) an aerosolisable formulation, comprising (i) caffeine; (ii) water; and (iii) propylene glycol; wherein the weight ratio of water to propylene glycol is from 30:70 to 60:40.

In one aspect there is provided an electronic aerosol provision system comprising: (a) an aerosoliser for aerosolising formulation for inhalation by a user of the electronic aerosol provision system; (b) a power supply comprising a cell or battery for supplying power to the aerosoliser (c) an aerosolisable formulation, comprising (i) caffeine; (ii) water; and (iii) propylene glycol; wherein the weight ratio of water to propylene glycol is from 30:70 to 60:40.

In one aspect there is provided a process for improving the solubility of caffeine in an aerosolisable formulation, the process comprising the steps of (a) providing an aerosolisable material comprising water and propylene glycol; (b) incorporating caffeine into the aerosolisable material to provide the aerosolisable formulation; wherein the weight ratio of water to propylene glycol is from 30:70 to 60:40.

In one aspect there is provided an electronic aerosol provision system comprising:
(a) an aerosoliser for aerosolising formulation for inhalation by a user of the electronic aerosol provision system;
(b) a power supply comprising a cell or battery for supplying power to the aerosoliser
(c) an aerosolisable formulation comprising caffeine;
wherein the aerosol provision system provides at least 0.5 mg of caffeine per gram of aerosol.

In one aspect there is provided use of water and propylene glycol to improve the solubility of caffeine in an aerosolisable formulation; wherein the weight ratio of water to propylene glycol is from 30:70 to 60:40.

### BRIEF DESCRIPTION OF THE DRAWINGS

The present invention will now be described in further detail by way of example only with reference to the accompanying figure in which:

FIG. 1 is highly schematic diagram (not to scale) of an example aerosol provision system; and

FIG. 2 shows caffeine solubility with cubic model for caffeine solubility in propylene glycol, glycerin, and water.

### DETAILED DESCRIPTION

As discussed herein in one aspect there is provided an aerosolisable formulation comprising (i) caffeine; (ii) water; and (iii) propylene glycol; wherein the weight ratio of water to propylene glycol is from 30:70 to 60:40.

We have found that an advantageous system may be provided which contains caffeine and in which the solvent is at least water and propylene glycol wherein the weight ratio of water to propylene glycol is from 30:70 to 60:40. In particular, we have identified that the solubility of caffeine in an aerosoliser formulation may be improved compared to typical systems containing propylene glycol and glycerol by dissolving the caffeine in a combination of at least water and propylene glycol wherein the water and propylene glycol are present in particular amounts relative to each other, namely a weight ratio of water to propylene glycol is from 30:70 to 60:40.

For ease of reference, these and further aspects of the present invention are now discussed under appropriate section headings. However, the teachings under each section are not necessarily limited to each particular section.

### Caffeine

Caffeine (1,3,7-trimethylpurine-2,6-dione) is a well-known stimulant. Around thirty plant species are known to contain caffeine. Common sources are the “beans” (seeds) of the two cultivated coffee plants, Coffea arabica and Coffea canephora, and of the cocoa plant Theobroma cacao; the leaves of the tea plant; and kola nuts.

Caffeine formulations may be provided having desirable properties and stimulant effect for the user. In one aspect caffeine is present in an amount of no greater than 20 wt % based on the total weight of the aerosolisable formulation. In one aspect caffeine is present in an amount of no greater than 15 wt % based on the total weight of the aerosolisable formulation. In one aspect caffeine is present in an amount of no greater than 10 wt % based on the total weight of the aerosolisable formulation. In one aspect caffeine is present in an amount of no greater than 5 wt % based on the total weight of the aerosolisable formulation. In one aspect caffeine is present in an amount of no greater than 3 wt % based on the total weight of the aerosolisable formulation. In one aspect caffeine is present in an amount of no greater than 2 wt % based on the total weight of the aerosolisable formulation. In one aspect caffeine is present in an amount of no greater than 1 wt % based on the total weight of the aerosolisable formulation.

In one aspect caffeine is present in an amount of at least 0.01 wt % based on the total weight of the aerosolisable formulation. In one aspect caffeine is present in an amount of at least 0.02 wt % based on the total weight of the aerosolisable formulation. In one aspect caffeine is present in an amount of at least 0.05 wt % based on the total weight of the aerosolisable formulation. In one aspect caffeine is present in an amount of at least 1 wt % based on the total weight of the aerosolisable formulation. In one aspect caffeine is present in an amount of at least 1.5 wt % based on the total weight of the aerosolisable formulation. In one aspect caffeine is present in an amount of at least 2 wt % based on the total weight of the aerosolisable formulation. In one aspect caffeine is present in an amount of at least 3 wt % based on the total weight of the aerosolisable formulation.

In one aspect caffeine is present in an amount of no greater than 10 wt % based on the total weight of the aerosolisable formulation. In one aspect caffeine is present in an amount of from 0.01 to 10 wt % based on the total weight of the aerosolisable formulation. In one aspect caffeine is present in an amount of from 0.02 to 10 wt % based on the total weight of the aerosolisable formulation. In one aspect caffeine is present in an amount of from 0.05 to 10 wt % based on the total weight of the aerosolisable formulation. In one aspect caffeine is present in an amount of from 0.08 to 10 wt % based on the total weight of the aerosolisable formulation.

In one aspect caffeine is present in an amount of no greater than 5 wt % based on the total weight of the aerosolisable formulation. In one aspect caffeine is present in an amount of from 0.01 to 5 wt % based on the total weight of the aerosolisable formulation. In one aspect caffeine is present in an amount of from 0.02 to 5 wt % based on the total weight of the aerosolisable formulation. In one aspect caffeine is present in an amount of from 0.05 to 5 wt % based on the total weight of the aerosolisable formulation. In one aspect caffeine is present in an amount of from 0.08 to 5 wt % based on the total weight of the aerosolisable formulation.

In one aspect caffeine is present in an amount of no greater than 4 wt % based on the total weight of the aerosolisable formulation. In one aspect caffeine is present in an amount of from 0.01 to 4 wt % based on the total weight of the aerosolisable formulation. In one aspect caffeine is present in an amount of from 0.02 to 4 wt % based on the total weight of the aerosolisable formulation. In one aspect caffeine is present in an amount of from 0.05 to 4 wt % based on the total weight of the aerosolisable formulation. In one aspect caffeine is present in an amount of from 0.08 to 4 wt % based on the total weight of the aerosolisable formulation.

In one aspect caffeine is present in an amount of no greater than 3 wt % based on the total weight of the aerosolisable formulation. In one aspect caffeine is present in an amount of from 0.01 to 3 wt % based on the total weight of the aerosolisable formulation. In one aspect caffeine is present in an amount of from 0.02 to 3 wt % based on the total weight of the aerosolisable formulation. In one aspect caffeine is present in an amount of from 0.05 to 3 wt % based on the total weight of the aerosolisable formulation. In one aspect caffeine is present in an amount of from 0.08 to 3 wt % based on the total weight of the aerosolisable formulation. In one aspect caffeine is present in an amount of from 0.1 to 3 wt % based on the total weight of the aerosolisable formulation. In one aspect caffeine is present in an amount of from 0.2 to 3 wt % based on the total weight of the aerosolisable formulation. In one aspect caffeine is present in an amount of from 0.5 to 3 wt % based on the total weight of the aerosolisable formulation. In one aspect caffeine is present in an amount of from 0.8 to 3 wt % based on the total weight of the aerosolisable formulation. In one aspect caffeine is present in an amount of from 1 to 3 wt % based on the total weight of the aerosolisable formulation. In one aspect caffeine is present in an amount of from 1.5 to 3 wt % based on the total weight of the aerosolisable formulation. In one aspect caffeine is present in an amount of from 2 to 3 wt % based on the total weight of the aerosolisable formulation.

In one aspect caffeine is present in an amount of no greater than 2 wt % based on the total weight of the aerosolisable formulation. In one aspect caffeine is present in an amount of from 0.01 to 2 wt % based on the total weight of the aerosolisable formulation. In one aspect caffeine is present in an amount of from 0.02 to 2 wt % based on the total weight of the aerosolisable formulation. In one aspect caffeine is present in an amount of from 0.05 to 2 wt % based on the total weight of the aerosolisable formulation. In one aspect caffeine is present in an amount of from 0.08 to 2 wt % based on the total weight of the aerosolisable formulation.

In one aspect caffeine is present in an amount of no greater than 1 wt % based on the total weight of the aerosolisable formulation. In one aspect caffeine is present in an amount of from 0.01 to 1 wt % based on the total weight of the aerosolisable formulation. In one aspect caffeine is present in an amount of from 0.02 to 1 wt % based on the total weight of the aerosolisable formulation. In one aspect caffeine is present in an amount of from 0.05 to 1 wt % based on the total weight of the aerosolisable formulation. In one aspect caffeine is present in an amount of from 0.08 to 1 wt % based on the total weight of the aerosolisable formulation. In one aspect caffeine is present in an amount of from 0.1 to 1 wt % based on the total weight of the aerosolisable formulation.

### Water

Water may be present in any suitable amount. In one aspect water is present in an amount of at least 5 wt. % based on the aerosolisable formulation. In one aspect water is present in an amount of at least 10 wt. % based on the aerosolisable formulation. In one aspect water is present in an amount of at least 15 wt. % based on the aerosolisable formulation. In one aspect water is present in an amount of at least 20 wt. % based on the aerosolisable formulation. In one aspect water is present in an amount of at least 25 wt. % based on the aerosolisable formulation. In one aspect water is present in an amount of at least 30 wt. % based on the aerosolisable formulation. In one aspect water is present in an amount of at least 35 wt. % based on the aerosolisable formulation.

In one aspect water is present in an amount of no greater than 70 wt. % based on the aerosolisable formulation. In one aspect water is present in an amount of no greater than 65 wt. % based on the aerosolisable formulation. In one aspect water is present in an amount of no greater than 60 wt. % based on the aerosolisable formulation. In one aspect water is present in an amount of no greater than 55 wt. % based on the aerosolisable formulation. In one aspect water is present in an amount of no greater than 50 wt. % based on the aerosolisable formulation. In one aspect water is present in an amount of no greater than 45 wt. % based on the aerosolisable formulation. In one aspect water is present in an amount of no greater than 40 wt. % based on the aerosolisable formulation.

In one aspect water is present in an amount of from 5 to 60 wt. % based on the aerosolisable formulation. In one aspect water is present in an amount of from 10 to 60 wt. % based on the aerosolisable formulation. In one aspect water is present in an amount of from 15 to 60 wt. % based on the aerosolisable formulation. In one aspect water is present in an amount of from 20 to 60 wt. % based on the aerosolisable formulation. In one aspect water is present in an amount of from 25 to 60 wt. % based on the aerosolisable formulation. In one aspect water is present in an amount of from 30 to 60 wt. % based on the aerosolisable formulation. In one aspect water is present in an amount of from 35 to 60 wt. % based on the aerosolisable formulation.

In one aspect water is present in an amount of from 5 to 50 wt. % based on the aerosolisable formulation. In one aspect water is present in an amount of from 10 to 50 wt. % based on the aerosolisable formulation. In one aspect water is present in an amount of from 15 to 50 wt. % based on the aerosolisable formulation. In one aspect water is present in an amount of from 20 to 50 wt. % based on the aerosolisable formulation. In one aspect water is present in an amount of from 25 to 50 wt. % based on the aerosolisable formulation. In one aspect water is present in an amount of from 30 to 50 wt. % based on the aerosolisable formulation. In one aspect water is present in an amount of from 35 to 50 wt. % based on the aerosolisable formulation.

In one aspect water is present in an amount of from 5 to 40 wt. % based on the aerosolisable formulation. In one aspect water is present in an amount of from 10 to 40 wt. % based on the aerosolisable formulation. In one aspect water is present in an amount of from 15 to 40 wt. % based on the aerosolisable formulation. In one aspect water is present in an amount of from 20 to 40 wt. % based on the aerosolisable formulation. In one aspect water is present in an amount of from 25 to 40 wt. % based on the aerosolisable formulation. In one aspect water is present in an amount of from 30 to 40 wt. % based on the aerosolisable formulation. In one aspect water is present in an amount of from 35 to 40 wt. % based on the aerosolisable formulation.

### Propylene Glycol

Propylene glycol may be present in any suitable amount. In one aspect propylene glycol is present in an amount of at least 5 wt. % based on the aerosolisable formulation. In one aspect propylene glycol is present in an amount of at least 10 wt. % based on the aerosolisable formulation. In one aspect propylene glycol is present in an amount of at least 15 wt. % based on the aerosolisable formulation. In one aspect propylene glycol is present in an amount of at least 20 wt. % based on the aerosolisable formulation. In one aspect propylene glycol is present in an amount of at least 25 wt. % based on the aerosolisable formulation. In one aspect propylene glycol is present in an amount of at least 30 wt. % based on the aerosolisable formulation. In one aspect propylene glycol is present in an amount of at least 35 wt. % based on the aerosolisable formulation. In one aspect propylene glycol is present in an amount of at least 40 wt. % based on the aerosolisable formulation.

In one aspect propylene glycol is present in an amount of no greater than 70 wt. % based on the aerosolisable formulation. In one aspect propylene glycol is present in an amount of no greater than 65 wt. % based on the aerosolisable formulation. In one aspect propylene glycol is present in an amount of no greater than 60 wt. % based on the aerosolisable formulation. In one aspect propylene glycol is present in an amount of no greater than 55 wt. % based on the aerosolisable formulation. In one aspect propylene glycol is present in an amount of no greater than 50 wt. % based on the aerosolisable formulation. In one aspect propylene glycol is present in an amount of no greater than 45 wt. % based on the aerosolisable formulation.

In one aspect propylene glycol is present in an amount of from 5 to 70 wt. % based on the aerosolisable formulation. In one aspect propylene glycol is present in an amount of from 10 to 70 wt. % based on the aerosolisable formulation. In one aspect propylene glycol is present in an amount of from 15 to 70 wt. % based on the aerosolisable formulation. In one aspect propylene glycol is present in an amount of from 20 to 70 wt. % based on the aerosolisable formulation. In one aspect propylene glycol is present in an amount of from 25 to 70 wt. % based on the aerosolisable formulation. In one aspect propylene glycol is present in an amount of from 30 to 70 wt. % based on the aerosolisable formulation. In one aspect propylene glycol is present in an amount of from 35 to 70 wt. % based on the aerosolisable formulation. In one aspect propylene glycol is present in an amount of from 40 to 70 wt. % based on the aerosolisable formulation.

In one aspect propylene glycol is present in an amount of from 5 to 60 wt. % based on the aerosolisable formulation. In one aspect propylene glycol is present in an amount of from 10 to 60 wt. % based on the aerosolisable formulation. In one aspect propylene glycol is present in an amount of from 15 to 60 wt. % based on the aerosolisable formulation. In one aspect propylene glycol is present in an amount of from 20 to 60 wt. % based on the aerosolisable formulation. In one aspect propylene glycol is present in an amount of from 25 to 60 wt. % based on the aerosolisable formulation. In one aspect propylene glycol is present in an amount of from 30 to 60 wt. % based on the aerosolisable formulation. In one aspect propylene glycol is present in an amount of from 35 to 60 wt. % based on the aerosolisable formulation. In one aspect propylene glycol is present in an amount of from 40 to 60 wt. % based on the aerosolisable formulation.

In one aspect propylene glycol is present in an amount of from 5 to 50 wt. % based on the aerosolisable formulation. In one aspect propylene glycol is present in an amount of from 10 to 50 wt. % based on the aerosolisable formulation. In one aspect propylene glycol is present in an amount of from 15 to 50 wt. % based on the aerosolisable formulation. In one aspect propylene glycol is present in an amount of from 20 to 50 wt. % based on the aerosolisable formulation. In one aspect propylene glycol is present in an amount of from 25 to 50 wt. % based on the aerosolisable formulation. In one aspect propylene glycol is present in an amount of from 30 to 50 wt. % based on the aerosolisable formulation. In one aspect propylene glycol is present in an amount of from 35 to 50 wt. % based on the aerosolisable formulation. In one aspect propylene glycol is present in an amount of from 40 to 50 wt. % based on the aerosolisable formulation.

### Water: Propylene Glycol

As discussed herein, the weight ratio of water to propylene glycol is from 30:70 to 60:40. As will be appreciated by one skilled in the art the weight ratio of water to propylene glycol is provided relative to each of the two materials and is irrespective of the presence of other materials.

In one aspect the weight ratio of water to propylene glycol is from 35:65 to 60:40. In one aspect the weight ratio of water to propylene glycol is from 40:60 to 60:40. In one aspect the weight ratio of water to propylene glycol is from 45:55 to 60:40.

In one aspect the weight ratio of water to propylene glycol is from 30:70 to 55:45. In one aspect the weight ratio of water to propylene glycol is from 30:70 to 50:50. In one aspect the weight ratio of water to propylene glycol is from 30:70 to 45:55.

In one aspect the weight ratio of water to propylene glycol is from 35:65 to 55:45. In one aspect the weight ratio of water to propylene glycol is from 40:60 to 50:50. In one aspect the weight ratio of water to propylene glycol is from 40:60 to 45:55.

### Glycerol

In one aspect, the aerosolisable formulation further comprises glycerol. Glycerol may be included in the aerosolisable formulation for a number of reasons. For example, the provision of a visible aerosol “cloud” when a formulation is exhaled by a user is desirable for many users. The presence of glycerol in an aerosolisable formulation may allow for a more visible effect.

When present, glycerol may be present in any suitable amount. In one aspect glycerol is present in an amount of at least 1 wt. % based on the aerosolisable formulation. In one aspect glycerol is present in an amount of at least 2 wt. % based on the aerosolisable formulation. In one aspect glycerol is present in an amount of at least 3 wt. % based on the aerosolisable formulation. In one aspect glycerol is present in an amount of at least 4 wt. % based on the aerosolisable formulation. In one aspect glycerol is present in an amount of at least 5 wt. % based on the aerosolisable formulation. In one aspect glycerol is present in an amount of at least 10 wt. % based on the aerosolisable formulation. In one aspect glycerol is present in an amount of at least 15 wt. % based on the aerosolisable formulation. In one aspect glycerol is present in an amount of at least 20 wt. % based on the aerosolisable formulation.

In one aspect glycerol is present in an amount of no greater than 40 wt. % based on the aerosolisable formulation. In one aspect glycerol is present in an amount of no greater than 35 wt. % based on the aerosolisable formulation. In one aspect glycerol is present in an amount of no greater than 30 wt. % based on the aerosolisable formulation. In one aspect glycerol is present in an amount of no greater than 25 wt. % based on the aerosolisable formulation. In one aspect glycerol is present in an amount of no greater than 20 wt. % based on the aerosolisable formulation. In one aspect glycerol is present in an amount of no greater than 15 wt. % based on the aerosolisable formulation. In one aspect glycerol is present in an amount of no greater than 10 wt. % based on the aerosolisable formulation.

In one aspect glycerol is present in an amount of from 1 to 60 wt. % based on the aerosolisable formulation. In one aspect glycerol is present in an amount of from 2 to 60 wt. % based on the aerosolisable formulation. In one aspect glycerol is present in an amount of from 5 to 60 wt. % based on the aerosolisable formulation. In one aspect glycerol is present in an amount of from 10 to 60 wt. % based on the aerosolisable formulation.

In one aspect glycerol is present in an amount of from 1 to 40 wt. % based on the aerosolisable formulation. In one aspect glycerol is present in an amount of from 2 to 40 wt. % based on the aerosolisable formulation. In one aspect glycerol is present in an amount of from 5 to 40 wt. % based on the aerosolisable formulation. In one aspect glycerol is present in an amount of from 10 to 40 wt. % based on the aerosolisable formulation.

In one aspect glycerol is present in an amount of from 1 to 20 wt. % based on the aerosolisable formulation. In one aspect glycerol is present in an amount of from 2 to 20 wt. % based on the aerosolisable formulation. In one aspect glycerol is present in an amount of from 5 to 20 wt. % based on the aerosolisable formulation. In one aspect glycerol is present in an amount of from 10 to 20 wt. % based on the aerosolisable formulation.

Despite the desirability of a visible aerosol “cloud” we have found that the presence of glycerol may be detrimental to the solubility of caffeine. Therefore, in some aspects, glycerol is present in restricted amounts or is not present at all. In one aspect the aerosolisable formulation contains glycerol in an amount of no greater than 10 wt. % based on the aerosolisable formulation. In one aspect the aerosolisable formulation contains glycerol in an amount of no greater than 8 wt. % based on the aerosolisable formulation. In one aspect the aerosolisable formulation contains glycerol in an amount of no greater than 5 wt. % based on the aerosolisable formulation. In one aspect the aerosolisable formulation contains glycerol in an amount of no greater than 2 wt. % based on the aerosolisable formulation. In one aspect the aerosolisable formulation contains glycerol in an amount of no greater than 1 wt. % based on the aerosolisable formulation. In one aspect the aerosolisable formulation contains glycerol in an amount of no greater than 0.5 wt. % based on the aerosolisable formulation. In one aspect the aerosolisable formulation contains glycerol in an amount of no greater than 0.2 wt. % based on the aerosolisable formulation. In one aspect the aerosolisable formulation contains glycerol in an amount of no greater than 0.1 wt. % based on the aerosolisable formulation. In one aspect the aerosolisable formulation contains glycerol in an amount of no greater than 0.01 wt. % based on the aerosolisable formulation. In one aspect the aerosolisable formulation contains no glycerol.

### Formulation

In one aspect the aerosolisable formulation further comprises one or more flavours or flavouring components. As used herein, the terms “flavour” and “flavourant” refer to materials which, where local regulations permit, may be used to create a desired taste or aroma in a product for adult consumers. They may include extracts (e.g. liquorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder. The one or more flavours may be selected from the group consisting of (4-(para-)methoxyphenyl)-2-butanone, vanillin, γ-undecalactone, menthone, 5-propenyl guaethol, menthol, para-mentha-8-thiol-3-one and mixtures thereof. In one aspect the flavour is at least menthol.

If present, the one or more flavours may be present in any suitable amount. In one aspect the one or more flavours are present in a total amount of no greater than 10 wt. % based on the aerosolisable formulation. In one aspect the one or more flavours are present in a total amount of no greater than 7 wt. % based on the aerosolisable formulation. In one aspect the one or more flavours are present in a total amount of no greater than 5 wt. % based on the aerosolisable formulation. In one aspect the one or more flavours are present in a total amount of no greater than 4 wt. % based on the aerosolisable formulation. In one aspect the one or more flavours are present in a total amount of no greater than 3 wt. % based on the aerosolisable formulation. In one aspect the one or more flavours are present in a total amount of no greater than 2 wt. % based on the aerosolisable formulation. In one aspect the one or more flavours are present in a total amount of no greater than 1 wt. % based on the aerosolisable formulation.

In one aspect the one or more flavours are present in a total amount of from 0.01 to 5 wt. % based on the aerosolisable formulation. In one aspect the one or more flavours are present in a total amount of from 0.01 to 4 wt. % based on the aerosolisable formulation. In one aspect the one or more flavours are present in a total amount of from 0.01 to 3 wt. % based on the aerosolisable formulation. In one aspect the one or more flavours are present in a total amount of from 0.01 to 2 wt. % based on the aerosolisable formulation. In one aspect the one or more flavours are present in a total amount of from 0.01 to 1 wt. % based on the aerosolisable formulation. In one aspect the one or more flavours are present in a total amount of from 0.01 to 0.5 wt. % based on the aerosolisable formulation.

In one aspect the aerosolisable formulation further comprises one or more solubilising agents. As used herein, the term “solubilising agent” refers to materials which increase the solubility of caffeine in the formulation. In one aspect, the one or more solubilising agents is selected from the group consisting of benzyl alcohol, triacetin, and mixtures thereof. In one aspect, the one or more solubilising agents is at least benzyl alcohol.

In one aspect the aerosolisable formulation is in liquid form. In one aspect the aerosolisable formulation is not in liquid form. In one aspect if the aerosolisable formulation is in the form of a gel or paste.

### Apparatus and Process

As discussed herein, in one aspect there is provided a process for forming an aerosol, the process comprising aerosolising an aerosolisable formulation comprising (i) caffeine; (ii) water; and (iii) propylene glycol; wherein the weight ratio of water to propylene glycol is from 30:70 to 60:40.

The formulation may be contained or delivered by any means. In one aspect the present invention provides a contained aerosolisable formulation comprising (a) one or more containers; and (b) an aerosolisable formulation as defined herein. The container may be any suitable container, for example to allow for the storage or delivery of the formulation. In one aspect the container is configured for engagement with an electronic aerosol provision system. The container may be configured to become fluidly in communication with an electronic aerosol provision system so that formulation may be delivered to the electronic aerosol provision system. As described above, the present disclosure relates to container which may be used in an electronic aerosol provision system, such as an e-cigarette. Throughout the following description the term “e-cigarette” is used; however, this term may be used interchangeably with electronic aerosol provision system.

As discussed herein, the container of the present invention is typically provided for the delivery of aerosolisable formulation to or within an e-cigarette. The aerosolisable formulation may be held within an e-cigarette or may be sold as a separate container for subsequent use with or in an e-cigarette. As understood by one skilled in the art, e-cigarettes may contain a unit known as a detachable cartomiser which typically comprises a reservoir of aerosolisable formulation, and an aerosoliser such as a wick material and a heating element for vaporising the aerosolisable formulation. In some e-cigarettes, the cartomiser is part of a single-piece device and is not detachable. In one aspect the container is a cartomiser or is part of a cartomiser. In one aspect the container is not a cartomiser or part of a cartomiser and is a container, such as a tank, which may be used to deliver nicotine formulation to or within an e-cigarette.

In one aspect the container is part of an e-cigarette. Therefore in a further aspect the present invention provides an electronic aerosol provision system comprising: an aerosolisable formulation as defined herein; an aerosoliser for aerosolising formulation for inhalation by a user of the electronic aerosol provision system; and a power supply comprising a cell or battery for supplying power to the aerosoliser.

In one aspect the present invention provides an electronic aerosol provision system comprising: (a) an aerosoliser for aerosolising formulation for inhalation by a user of the electronic aerosol provision system; (b) a power supply comprising a cell or battery for supplying power to the aerosoliser; and (c) an aerosolisable formulation comprising caffeine; wherein the aerosol provision system provides at least 0.5 mg of caffeine per gram of aerosol. We have found that the present system allows for the delivery of particularly high amounts of caffeine per gram of aerosol. This is at least in part due to high solubility of caffeine in the aerosolisable formulation.

In one aspect the aerosol provision system provides at least 1 mg of caffeine per gram of aerosol. In one aspect the aerosol provision system provides at least 2 mg of caffeine per gram of aerosol. In one aspect the aerosol provision system provides at least 5 mg of caffeine per gram of aerosol. In one aspect the aerosol provision system provides at least 10 mg of caffeine per gram of aerosol. In one aspect the aerosol provision system provides at least 15 mg of caffeine per gram of aerosol. In one aspect the aerosol provision system provides at least 20 mg of caffeine per gram of aerosol. In one aspect the aerosol provision system provides at least 25 mg of caffeine per gram of aerosol. In one aspect the aerosol provision system provides at least 30 mg of caffeine per gram of aerosol. In one aspect the aerosol provision system provides at least 40 mg of caffeine per gram of aerosol.

In one aspect the aerosol provision system provides 0.5 to 100 mg of caffeine per gram of aerosol. In one aspect the aerosol provision system provides 0.5 to 90 mg of caffeine per gram of aerosol. In one aspect the aerosol provision system provides 0.5 to 80 mg of caffeine per gram of aerosol. In one aspect the aerosol provision system provides 0.5 to 70 mg of caffeine per gram of aerosol. In one aspect the aerosol provision system provides 0.5 to 60 mg of caffeine per gram of aerosol. In one aspect the aerosol provision system provides 0.5 to 50 mg of caffeine per gram of aerosol. In one aspect the aerosol provision system provides 1 to 50 mg of caffeine per gram of aerosol. In one aspect the aerosol provision system provides 2 to 50 mg of caffeine per gram of aerosol. In one aspect the aerosol provision system provides 5 to 50 mg of caffeine per gram of aerosol. In one aspect the aerosol provision system provides 10 to 50 mg of caffeine per gram of aerosol. In one aspect the aerosol provision system provides 15 to 50 mg of caffeine per gram of aerosol. In one aspect the aerosol provision system provides 20 to 50 mg of caffeine per gram of aerosol. In one aspect the aerosol provision system provides 25 to 50 mg of caffeine per gram of aerosol. In one aspect the aerosol provision system provides 30 to 50 mg of caffeine per gram of aerosol. In one aspect the aerosol provision system provides 30 to 40 mg of caffeine per gram of aerosol.

In addition to the aerosolisable formulation of the present invention and to systems such as containers and electronic aerosol provision systems containing the same, the present invention provides a process for improving the solubility of caffeine in an aerosolisable formulation, the process comprising the steps of (a) providing an aerosolisable material comprising water and propylene glycol; (b) incorporating caffeine into the aerosolisable material to provide the aerosolisable formulation; wherein the weight ratio of water to propylene glycol is from 30:70 to 60:40.

In a further aspect there is provided an article comprising the aerosolisable formulation as defined herein. The article may be a container, such as a bottle, or may be a component for use with an aerosol provision device. For example, the article may comprise an area (store) for receiving the aerosolisable formulation defined herein, an aerosol generating component, an aerosol generating area, and/or a mouthpiece.

In some embodiments, there is provided an article for use with an aerosol provision system, the article comprising a store comprising an aerosolisable formulation as defined herein, an aerosol generating component (such as a heater), an aerosol generating area, a transport element, and a mouthpiece.

Aerosolisable formulation may be transferred from the store for receiving an aerosolisable formulation to the aerosol generating component via a transport element, such as a wick, pump or the like. The skilled person is able to select suitable transport elements depending on the type of aerosolisable formulation that is to be transported and the rate at which it must be supplied. Particular mention may be made of transport elements, such as wicks, formed from fibrous materials, foamed materials, sintered materials, woven and non-woven materials.

An airflow pathway typically extends through the article (optionally via the device) to an outlet. The pathway is oriented such that generated aerosol is entrained in the airflow such that it can be delivered to the outlet for inhalation by a user.

In one embodiment, the aerosol generating component is a heater.

In one embodiment, the aerosol generating component applies ultrasonic energy to the aerosolisable formulation. The aerosol may be formed at a temperature below 60° C. The aerosol may be formed at a temperature below 50° C. The aerosol may be formed at a temperature below 40° C. The aerosol may be formed at a temperature below 30° C. The aerosol may be formed at a temperature below 25° C.

Typically, the area for receiving an aerosolisable formulation will allow for the article to be refilled with aerosolisable formulation as the aerosolisable formulation is depleted during use.

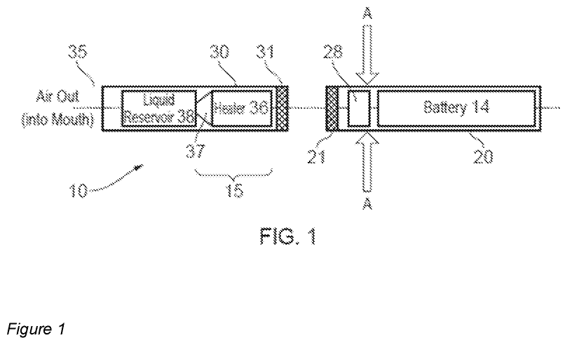

FIG. 1 is a highly schematic diagram (not to scale) of an example aerosol provision system, such as an e-cigarette 10, to which embodiments are applicable. The e-cigarette has a generally cylindrical shape, extending along a longitudinal axis indicated by a dashed line (although aspects of the invention are applicable to e-cigarettes configured in other shapes and arrangements), and comprises two main components, namely an aerosol provision device 20 and an article 30.

The article 30 includes a store for aerosolisable formulation (source liquid) 38 containing an aerosolisable formulation (source liquid) from which an aerosol is to be generated. The article 30 further comprises an aerosol generating component (heating element or heater) 36 for heating aerosolisable formulation to generate the aerosol. A transport element or wicking element or wick 37 is provided to deliver aerosolisable formulation from the store 38 to the heating element 36. A part or parts of the wick 37 are in fluid communication with aerosolisable formulation in the store 38 and by a wicking or capillary action aerosolisable formulation is drawn along or through the wick 37 to a part or parts of the wick 37 which are in contact with the heater 36.

Vaporization of the aerosolisable formulation occurs at the interface between the wick 37 and the heater 36 by the provision of heat energy to the aerosolisable formulation to cause evaporation, thus generating the aerosol. The aerosolisable formulation, the wick 37 and the heater 36 may be collectively referred to as an aerosol or vapour source. The wick 37 and the heater 36 may be collectively referred to as a vaporizer or an atomiser 15.

The article 30 further includes a mouthpiece 35 having an opening through which a user may inhale the aerosol generated by the vaporizer 15. The aerosol for inhalation may be described as an aerosol stream or inhalable airstream.

The aerosol delivery device 20 includes a power source (a re-chargeable cell or battery 14, referred to herein after as a battery) to provide power for the e-cigarette 10, and a controller (printed circuit board (PCB)) 28 and/or other electronics for generally controlling the e-cigarette 10. The aerosol delivery device can therefore also be considered as a battery section, or a control unit or section.

For example, in use, when the heater 36 receives power from the battery 14, as controlled by the circuit board 28 possibly in response to pressure changes detected by an air pressure sensor (not shown), the heater 36 vaporizes aerosolisable formulation delivered by the wick 37 to generate the aerosol, and this aerosol stream is then inhaled by a user through the opening in the mouthpiece 35. The aerosol is carried from the aerosol source to the mouthpiece 35 along an air channel (not shown in FIG. 1) that connects the aerosol source to the mouthpiece opening as a user inhales on the mouthpiece.

In a further aspect there is provided an aerosol provision system comprising an aerosol provision device and an article as defined herein.

In this regard, the present method may be further defined according to any of the features described above.

In a further aspect, there is provided a method for producing an aerosol comprising generating an aerosol from an aerosolisable formulation as defined herein.

The process of the present invention may comprises additional steps either before the steps listed, after the steps listed or between one or more of the steps listed.

### Further Aspects

In a further aspect there is provided an aerosolisable formulation comprising (i) caffeine; (ii) water in an amount of from 10 to 60 wt. % based on the aerosolisable formulation; (iii) glycerol in an amount of at least 10 wt. % based on the aerosolisable formulation; and (iv) propylene glycol in an amount of at least 10 wt. % based on the aerosolisable formulation.

In a further aspect there is provided a process for forming an aerosol, the process comprising aerosolising an aerosolisable formulation comprising (i) caffeine; (ii) water in an amount of from 10 to 60 wt. % based on the aerosolisable formulation; (iii) glycerol in an amount of at least 10 wt. % based on the aerosolisable formulation; and (iv) propylene glycol in an amount of at least 10 wt. % based on the aerosolisable formulation.

In a further aspect there is provided a contained aerosolisable formulation comprising (a) a container; and (b) an aerosolisable formulation, comprising (i) caffeine; (ii) water in an amount of from 10 to 60 wt. % based on the aerosolisable formulation; (iii) glycerol in an amount of at least 10 wt. % based on the aerosolisable formulation; and (iv) propylene glycol in an amount of at least 10 wt. % based on the aerosolisable formulation.

In a further aspect there is provided an electronic aerosol provision system comprising: (a) an aerosoliser for aerosolising formulation for inhalation by a user of the electronic aerosol provision system; (b) a power supply comprising a cell or battery for supplying power to the aerosoliser (c) an aerosolisable formulation, comprising (i) caffeine; (ii) water in an amount of from 10 to 60 wt. % based on the aerosolisable formulation; (iii) glycerol in an amount of at least 10 wt. % based on the aerosolisable formulation; and (iv) propylene glycol in an amount of at least 10 wt. % based on the aerosolisable formulation.

In a further aspect there is provided a process for improving the solubility of caffeine in an aerosolisable formulation, the process comprising the steps of (a) providing an aerosolisable material comprising water, glycerol and propylene glycol; (b) incorporating caffeine into the aerosolisable material to provide the aerosolisable formulation;

wherein water is present in an amount of from 10 to 60 wt. % based on the aerosolisable formulation; glycerol is present in an amount of at least 10 wt. % based on the aerosolisable formulation; and propylene glycol is present in an amount of at least 10 wt. % based on the aerosolisable formulation.

The invention will now be described with reference to the following non-limiting examples.

### Examples

All percentages given in the examples are weight percentages.

An initial analysis was conducted of a typical aerosolisable formulation containing propylene glycol (PG) and glycerol. The solubility of caffeine across a range of amounts of these two components was determined. No water was added to the formulations.

TABLE 1

| | | | Measured |
|---|---|---|---|
| Formulation | % Glycerol | % PG | Concentration (mg/g) |
| 1.1 | 100 | 0 | 4.75 |
| 1.2 | 80 | 20 | 6.50 |
| 1.3 | 60 | 40 | 7.15 |
| 1.4 | 50 | 50 | 10.16 |
| 1.5 | 40 | 60 | 10.97 |
| 1.6 | 20 | 80 | 12.45 |
| 1.7 | 0 | 100 | 11.95 |

An analysis was then conducted of aerosolisable formulations containing propylene glycol (PG) and water. The solubility of caffeine across a range of amounts of these two components was determined. No glycerol was added to the formulations.

TABLE 2

| | | | Measured |
|---|---|---|---|
| Formulation | % PG | % Water | Concentration (mg/g) |
| 2.1 | 0 | 100 | 19.75 |
| 2.2 | 10 | 90 | 18.45 |
| 2.3 | 20 | 80 | 26.97 |
| 2.4 | 30 | 70 | 24.54 |
| 2.5 | 40 | 60 | 25.96 |
| 2.6 | 50 | 50 | 31.15 |
| 2.7 | 60 | 40 | 27.79 |
| 2.8 | 70 | 30 | 25.75 |
| 2.9 | 80 | 20 | 23.48 |
| 2.10 | 90 | 10 | 23.60 |
| 2.11 | 100 | 0 | 11.60 |

Formulations 2.5 to 2.8 are in accordance with the present invention and provided a formulation containing at least 25 mg of caffeine per g of formulation.

An analysis was also conducted of aerosolisable formulations containing propylene glycol (PG), water and glycerol. The solubility of caffeine across a range of amounts of these three components was determined. No glycerol was added to the formulations.

TABLE 3

| | | | | | Measured |
|---|---|---|---|---|---|
| | % | % | % | Water:PG | Concentration |
| Formulation | Glycerol | PG | Water | (wt.) | (mg/g) |
| 3.1 | 50 | 40 | 10 | 20:80 | 13.47 |
| 3.2 | 50 | 25 | 25 | 50:50 | 20.20 |
| 3.3 | 50 | 0 | 50 | 100:0 | 14.20 |
| 3.4 | 30 | 40 | 30 | 43:57 | 24.89 |
| 3.5 | 10 | 40 | 50 | 56:44 | 26.68 |
| 3.6 | 36 | 54 | 10 | 17:83 | 7.30 |
| 3.7 | 33 | 33 | 33 | 50:50 | 12.00 |
| 3.8 | 25 | 50 | 25 | 33:67 | 21.90 |
| 3.9 | 10 | 50 | 40 | 57:43 | 27.50 |
| 3.10 | 30 | 60 | 10 | 14:86 | 13.49 |
| 3.11 | 25 | 0 | 75 | 100:0 | 15.60 |
| 3.12 | 20 | 60 | 20 | 25:75 | 16.67 |

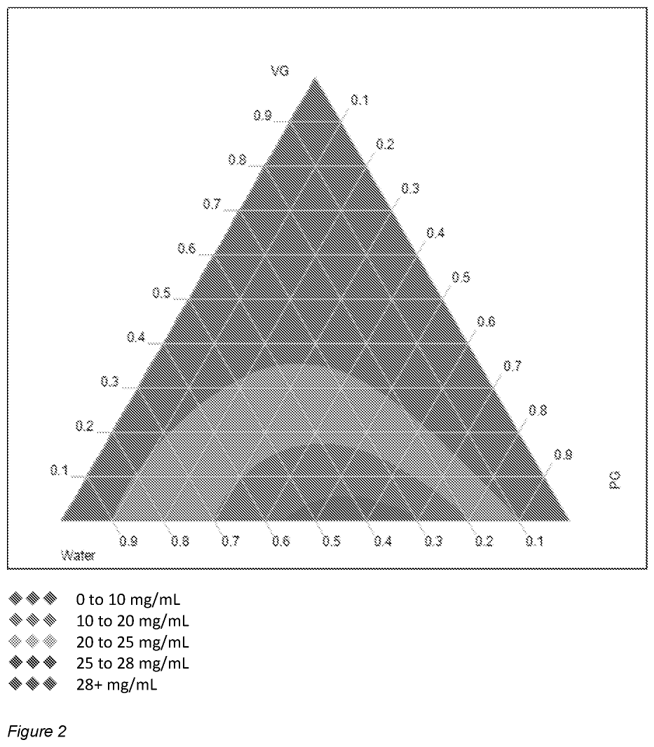

It was found that although the presence of glycerol reduced the overall solubility of caffeine, the formulation in accordance with the present invention provided optimum solubility of caffeine. The data of Table 3 are modelled in FIG. 2.

A number of the formulations, both flavoured and unflavoured, were tested in ePen3 and ePod devices available from British American Tobacco. The results are shown in Table 4.

TABLE 4

| | | | Caffeine | Caffeine | |
|---|---|---|---|---|---|
| | Glycerol/PG/Water | Water:PG | Concentration in | Concentration | Transfer |
| Sample Description | weight Ratio | weight Ratio | Formulation (mg/g) | in Aerosol (mg/g) | to Aerosol |
| 28 mg/mL caffeine, ePen3, unflavored | 8/49/43 | 47:53 | 27.80 | 20.8 | 75% |
| 28 mg/mL caffeine, ePen3, flavored | 8/49/43 | 47:53 | 27.50 | 21.8 | 79% |
| 28 mg/mL caffeine, ePod, unflavored | 8/49/43 | 47:53 | 27.80 | 21.7 | 78% |
| 28 mg/mL caffeine, ePod, flavored | 8/49/43 | 47:53 | 27.50 | 23.8 | 87% |
| 30 mg/mL caffeine, ePen3, coffee | 11/49/40 | 45:55 | 30.50 | 52.9 | * >100% |
| 30 mg/mL caffeine, ePod, coffee | 11/49/40 | 45:55 | 30.50 | 39.6 | * >100% |
| 3 mg/mL caffeine, ePod, unflavored | 50/50/0 | 0:100 | 3.00 | 2.3 | 77% |
| 3 mg/mL caffeine, ePod, flavored wild berry | 50/50/0 | 0:100 | 2.80 | 2.3 | 82% |
| 5 mg/mL caffeine, ePen3, unflavored | 36/54/10 | 16:84 | 5.11 | 4.2 | 82% |
| 5 mg/mL caffeine, ePen3, , wild berry | 36/54/10 | 16:84 | 5.30 | 4.9 | 92% |
| 27 mg/mL caffeine, iSwitch, unflavored | 0/50/50 | 50:50 | 26.9 | 18.8 | 69.8 |
| 26 mg/mL caffeine, iSwitch, flavored | 0/50/50 | 50:50 | 25.8 | 26.2 | 101.6 |
| 3 mg/mL caffeine, iSwitch, unflavored | 0/50/50 | 50:50 | 2.8 | 1.1 | 40.1 |
| 3 mg/mL caffeine, iSwitch, flavored | 0/50/50 | 50:50 | 2.7 | 0.9 | 33.2 |
| 3 mg/mL caffeine, iSwitch, unflavored | 63/36/1 | 3:97 | 2.7 | 2.3 | 83.3 |
| 3 mg/mL caffeine, iSwitch, flavored | 63/36/1 | 3:97 | 2.5 | 2.6 | 103.1 |

Further formulations were prepared containing solubilising agent, namely benzyl alcohol. Each of the formulations contained 30 mg of caffeine per ml of formulation.

TABLE 5

| Formulation | 4.1 | 4.2 | 4.3 | 4.4 |
|---|---|---|---|---|
| Glycerol (wt. %) | 10.0000 | 10.0000 | 10.0000 | 10.0000 |
| PG (wt. %) | 43.4964 | 44.5464 | 47.2464 | 43.4995 |
| Water (wt. %) | 35.2500 | 39.0000 | 39.0000 | 35.2468 |
| Flavour (wt. %) | 7.5000 | 2.7000 | 0.0000 | 7.5000 |
| Benzyl alcohol (wt. %) | 0.8984 | 0.8984 | 0.8984 | 0.8984 |
| Caffeine (wt. %) | 2.8552 | 2.8552 | 2.8552 | 2.8552 |
| Total (wt. %) | 100.0000 | 100.0000 | 100.0000 | 100.0000 |

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. An aerosolisable formulation comprising
(i) caffeine;
(ii) water; and
(iii) propylene glycol;
wherein the weight ratio of water to propylene glycol is from 30:70 to 60:40.

2. The aerosolisable formulation according to claim 1, wherein caffeine is present in an amount of no greater than 10 wt. % based on the aerosolisable formulation.

3. The aerosolisable formulation according to claim 1, wherein caffeine is present in an amount of no greater than 5 wt. % based on the aerosolisable formulation.

4. The aerosolisable formulation according to claim 1, wherein caffeine is present in an amount of no greater than 3 wt. % based on the aerosolisable formulation.

5. The aerosolisable formulation according to claim 1, wherein water is present in an amount of from 10 to 60 wt. % based on the aerosolisable formulation.

6. The aerosolisable formulation according to claim 1, wherein water is present in an amount of from 20 to 60 wt. % based on the aerosolisable formulation.

7. The aerosolisable formulation according to claim 1, wherein water is present in an amount of from 30 to 50 wt. % based on the aerosolisable formulation.

8. The aerosolisable formulation according to claim 1, further comprising glycerol.

9. The aerosolisable formulation according to claim 8, wherein glycerol is present in an amount of from 10 to 60 wt. % based on the aerosolisable formulation.

10. The aerosolisable formulation according to claim 8, wherein glycerol is present in an amount of from 10 to 40 wt. % based on the aerosolisable formulation.

11. The aerosolisable formulation according to claim 8, wherein glycerol is present in an amount of no greater than 20 wt. % based on the aerosolisable formulation.

12. The aerosolisable formulation according to claim 8, wherein glycerol is present in an amount of no greater than 10 wt. % based on the aerosolisable formulation.

13. The aerosolisable formulation according to claim 1, wherein propylene glycol is present in an amount of at least 10 wt. % based on the aerosolisable formulation.

14. The aerosolisable formulation according to claim 1, wherein propylene glycol is present in an amount of from 10 to 60 wt. % based on the aerosolisable formulation.

15. The aerosolisable formulation according to claim 1, wherein propylene glycol is present in an amount of from 20 to 50 wt. % based on the aerosolisable formulation.

16. The aerosolisable formulation according to claim 1, further comprising one or more flavours.

17. The aerosolisable formulation according to claim 16, wherein the one or more flavours are selected from the group consisting of (4-(para-)methoxyphenyl)-2-butanone, vanillin, γ-undecalactone, menthone, 5-propenyl guaethol, menthol, para-mentha-8-thiol-3-one and mixtures thereof.

18. The aerosolisable formulation according to claim 16, wherein the one or more flavours are present in a total amount of from 0.01 to 1 wt. % based on the aerosolisable formulation.

19. The aerosolisable formulation according to claim 1, further comprising one or more solubilising agents.

20. The aerosolisable formulation according to claim 19, wherein the one or more solubilising agents is selected from the group consisting of benzyl alcohol, triacetin, and mixtures thereof.

21. A process for forming an aerosol, the process comprising aerosolising an aerosolisable formulation comprising
(i) caffeine;
(ii) water; and
(iii) propylene glycol;
wherein the weight ratio of water to propylene glycol is from 30:70 to 60:40.

22. The process according to claim 21, wherein the aerosolisable formulation comprises caffeine in an amount of no greater than 10 wt. % based on the aerosolisable formulation.

23. A contained aerosolisable formulation comprising
(a) a container; and
(b) an aerosolisable formulation, comprising
(i) caffeine;
(ii) water; and
(iii) propylene glycol;
wherein the weight ratio of water to propylene glycol is from 30:70 to 60:40.

24. The contained aerosolisable formulation according to claim 23, wherein the aerosolisable formulation comprises caffeine in an amount of no greater than 10 wt. % based on the aerosolisable formulation.

25. The contained aerosolisable formulation according to claim 23, wherein the container is configured for engagement with an electronic aerosol provision system.

26. An electronic aerosol provision system comprising:
(a) an aerosoliser for aerosolising formulation for inhalation by a user of the electronic aerosol provision system;
(b) a power supply comprising a cell or battery for supplying power to the aerosoliser
(c) an aerosolisable formulation, comprising
(i) caffeine;
(ii) water; and
(iii) propylene glycol;
wherein the weight ratio of water to propylene glycol is from 30:70 to 60:40.

27. The electronic aerosol provision system according to claim 26, wherein the aerosolisable formulation comprises caffeine in an amount of no greater than 10 wt. % based on the aerosolisable formulation.

28. A process for improving the solubility of caffeine in an aerosolisable formulation, the process comprising the steps of
(a) providing an aerosolisable material comprising water and propylene glycol;
(b) incorporating caffeine into the aerosolisable material to provide the aerosolisable formulation;
wherein the weight ratio of water to propylene glycol is from 30:70 to 60:40.

29. The process according to claim 28, wherein the aerosolisable formulation comprises caffeine in an amount of no greater than 10 wt. % based on the aerosolisable formulation.

30. An electronic aerosol provision system comprising:
(a) an aerosoliser for aerosolising formulation for inhalation by a user of the electronic aerosol provision system;
(b) a power supply comprising a cell or battery for supplying power to the aerosoliser; and
(c) an aerosolisable formulation comprising caffeine;
wherein the aerosol provision system provides at least 0.5 mg of caffeine per gram of aerosol.

31. The electronic aerosol provision system according to claim 30, wherein the aerosol provision system provides 0.5 to 50 mg of caffeine per gram of aerosol.

32. The electronic aerosol provision system according to claim 30, wherein the aerosolisable formulation comprises (i) caffeine present in an amount of no greater than 10 wt. % based on the aerosolisable formulation; (ii) water; and (iii) propylene glycol; wherein the weight ratio of water to propylene glycol is from 30:70 to 60:40.
